(19) United States
(12) Patent Application Publication
(10) Pub. No.: US 2026/0155216 A1
(43) Pub. Date: Jun. 4, 2026

(54) CLINICAL TRIAL SUPPORT DEVICE, CLINICAL TRIAL SUPPORT METHOD, AND RECORDING MEDIUM

(71) Applicant: NEC Corporation, Tokyo (JP)

(72) Inventors: Daisaku SHIBATA, Tokyo (JP); Masanori Tsujikawa, Tokyo (JP); Masahiro Kubo, Tokyo (JP); Kei Shibuya, Tokyo (JP); Hiroki Goto, Tokyo (JP); Yoshiyasu Aoki, Tokyo (JP); Tomoko Morita, Tokyo (JP)

(73) Assignee: NEC Corporation, Tokyo (JP)

(21) Appl. No.: 19/398,568

(22) Filed: Nov. 24, 2025

(30) Foreign Application Priority Data
Nov. 29, 2024 (JP) 2024-208043

(51) Int. Cl. G16H 10/20; G16H 10/60

(52) U.S. Cl. CPC G16H 10/20; G16H 10/60

(57) ABSTRACT

A clinical trial support device includes an acquisition unit, an extraction unit, an estimation unit, a prediction unit, and an output unit. The extraction unit extracts, as a clinical trial candidate, a patient who satisfies eligibility criteria of the clinical trial patient at the first time point based on data regarding treatment of the patient. The estimation unit estimates a matching probability that is a probability that the clinical trial candidate is suitable for the clinical trial at the second time point by using a state curve that is a curve indicating a time-series state change of a patient suffering from the disease to be studied. The prediction unit predicts the number of clinical trial candidates at the second time point based on the matching probability. With such a configuration, the clinical trial support device can support a decision-making regarding implementation of a clinical trial.

This application is based upon and claims the benefit of priority from Japanese Patent Application No. 2024-208043, filed on Nov. 29, 2024, the disclosure of which is incorporated herein in its entirety by reference.

### TECHNICAL FIELD

The present disclosure relates to a clinical trial support device and the like.

### BACKGROUND ART

In a pharmaceutical trial, a system that supports selection of patients to be studied may be used to select patients to be studied.

The clinical study support system of WO 2023/248978 A1 screens a database storing clinical data based on eligibility criteria and exclusion criteria of a clinical study. Then, the clinical study support system of WO 2023/248978 A1 narrows down candidates for a clinical study based on a screening result.

### SUMMARY

A clinical trial support device according to an aspect of the present disclosure includes an acquisition unit that acquires data regarding treatment of a patient, an extraction unit that extracts, as a clinical trial candidate, a patient who satisfies eligibility criteria of a clinical trial patient at a first time point based on the data regarding the treatment, an estimation unit that estimates a matching probability that is a probability that the clinical trial candidate is suitable for the clinical trial at a second time point after the first time point by using a state curve that is a curve indicating a time-series state change of a patient suffering from a disease to be studied, a prediction unit that predicts the number of clinical trial candidates at the second time point based on the matching probability, and an output unit that outputs information regarding the number of clinical trial candidates at the second time point.

A clinical trial support method according to an aspect of the present disclosure includes: acquiring data regarding treatment of a patient, extracting, as a clinical trial candidate, a patient who satisfies eligibility criteria of a clinical trial patient at a first time point based on the data regarding the treatment, estimating a matching probability that is a probability that the clinical trial candidate is suitable for the clinical trial at a second time point after the first time point by using a state curve that is a curve indicating a time-series state change of a patient suffering from a disease to be studied, predicting the number of clinical trial candidates at the second time point based on the matching probability, and outputting information regarding the number of clinical trial candidates at the second time point.

A non-transitory recording medium according to an aspect of the present disclosure records a program for causing a computer to execute a process of acquiring data regarding treatment of a patient, a process of extracting, as a clinical trial candidate, a patient who satisfies eligibility criteria of a clinical trial patient at a first time point based on the data regarding the treatment, a process of estimating a matching probability that is a probability that the clinical trial candidate is suitable for the clinical trial at a second time point after the first time point by using a state curve that is a curve indicating a time-series state change of a patient suffering from a disease to be studied, a process of predicting the number of clinical trial candidates at the second time point based on the matching probability, and a process of outputting information regarding the number of clinical trial candidates at the second time point.

### BRIEF DESCRIPTION OF THE DRAWINGS

Exemplary features and advantages of the present disclosure will become apparent from the following detailed description when taken with the accompanying drawings in which:

FIG. 1 is a diagram illustrating an example of a configuration of a clinical trial support system in the present disclosure;

FIG. 2 is a diagram illustrating an example of a configuration of a clinical trial support device in the present disclosure;

FIG. 3 is a diagram schematically illustrating an example of a curve illustrating a state change in a patient in the present disclosure;

FIG. 4 is a diagram schematically illustrating an example of a curve illustrating a state change in a patient in the present disclosure;

FIG. 5 is a diagram illustrating an example of a display screen for displaying a prediction result of the number of clinical trial candidates in the present disclosure;

FIG. 6 is a diagram illustrating an example of a display screen for displaying a prediction result of the number of clinical trial candidates in the present disclosure;

FIG. 7 is a diagram illustrating an example of a display screen for displaying a prediction result of the number of clinical trial candidates in the present disclosure;

FIG. 8 is a diagram illustrating an example of a screen for setting conditions for extracting a clinical trial candidate in the present disclosure;

FIG. 9 is a diagram illustrating an example of a display screen for displaying a prediction result of the number of clinical trial candidates in the present disclosure;

FIG. 10 is a diagram illustrating an example of a display screen for displaying a prediction result of the number of clinical trial candidates in the present disclosure;

FIG. 11 is a diagram illustrating an example of a display screen for displaying a prediction result of the number of clinical trial candidates in the present disclosure;

FIG. 12 is a diagram illustrating an example of a display screen for displaying details of a clinical trial candidate in the present disclosure;

FIG. 13 is a diagram illustrating an example of an operation flow of a clinical trial support device in the present disclosure; and

FIG. 14 is a diagram illustrating an example of a hardware configuration of a clinical trial support device in the present disclosure.

### EXAMPLE EMBODIMENT

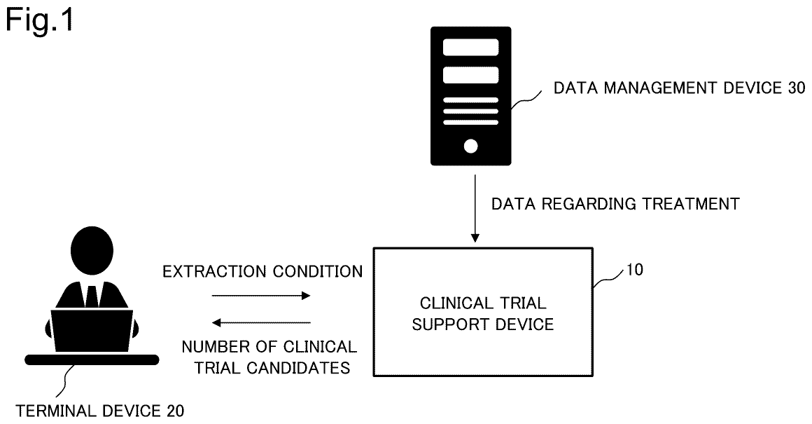

Example embodiments of the present disclosure will be described in detail with reference to the drawings. FIG. 1 is an example of a configuration of a clinical trial support system. The clinical trial support system includes a clinical trial support device 10, a terminal device 20, and a data management device 30. The clinical trial support device 10 is connected to the terminal device 20 via, for example, a network. The clinical trial support device 10 is connected to the data management device 30 via, for example, a network. A plurality of terminal devices 20 and a plurality of data management devices 30 may be provided. The number of terminal devices 20 and the number of data management devices 30 can be appropriately set.

The clinical trial support system is, for example, a system that predicts the number of clinical trial candidates. The clinical trial candidate is, for example, a candidate for a clinical trial patient. A clinical trial patient is, for example, a candidate for a patient to be administered a medicine in a clinical study of the medicine. Clinical trial patients may include control group patients. A pharmaceutical company or an institution commissioned by a pharmaceutical company creates a clinical trial implementation protocol, for example, by estimating the number of clinical trial patients at the stage of starting a clinical trial. Then, the pharmaceutical company or the institution commissioned by the pharmaceutical company receives an examination as to whether to implement a clinical trial based on the created clinical trial implementation protocol, for example. In a case where it is recognized in the examination that the clinical trial can be carried out, the pharmaceutical company or the institution commissioned by the pharmaceutical company carries out the clinical trial based on the clinical trial implementation protocol, for example, after securing the clinical trial patients. Since it takes time from the time of creation of the clinical trial implementation protocol to the start of the clinical trial, for example, even a patient who has satisfied the eligibility criteria of the clinical trial patient at the time of creation of the clinical trial implementation protocol may be in a state not suitable for the clinical trial at the start of the clinical trial. Therefore, in order to carry out the clinical trial as scheduled, for example, it is desirable that the number of clinical trial candidates at the start of the clinical trial can be accurately predicted at the time of creating the clinical trial implementation protocol.

For example, the clinical trial support system extracts a patient satisfying the eligibility criteria of the clinical trial patient as a clinical trial candidate based on data regarding treatment of the patient. The clinical trial support system estimates, for example, a probability that the extracted clinical trial candidate is in a state of being suitable as a clinical trial patient at the start of the clinical trial as a matching probability. Then, the clinical trial support system predicts the number of clinical trial candidates at the start of the clinical trial, for example, based on the estimated matching probability.

The clinical trial support system estimates the matching probability using, for example, a state curve. The state curve is, for example, a curve illustrating a time-series state change of a patient suffering from a disease to be studied. The state curve is, for example, a curve indicating the probability that a patient suffering from a disease to be studied is present in a state of being suitable for the clinical trial. The state of being suitable for the clinical trial is, for example, a state of being a subject to be administered with a medicine to be studied. The state of being a subject to be administered with a medicine to be studied is, for example, a state in which a patient is alive and has not finished treatment. The state of being a subject to be administered with a medicine to be studied is, for example, a state in which a patient is alive and another medicine having a similar purpose is not administered. As the state curve, for example, a Kaplan-Meier curve is used. The state curve is not limited to the above.

For example, assuming that the time of creation of the clinical trial implementation protocol is a first time point and the time of start of the clinical trial is a second time point, the clinical trial support system estimates, as a matching probability, a probability that a clinical trial candidate satisfying the eligibility criteria of the clinical trial patient at the first time point is matched as the clinical trial patient at the second time point. Then, the clinical trial support system predicts the number of clinical trial candidates at the second time point based on the estimated matching probability using, for example, the state curve. The first time point is not limited to the time point of creation of the clinical trial implementation protocol. Also, the second time point is not limited to the start time point of the clinical trial. In this way, by predicting the number of clinical trial candidates, the clinical trial support system can improve the accuracy of prediction of the number of clinical trial candidates, for example.

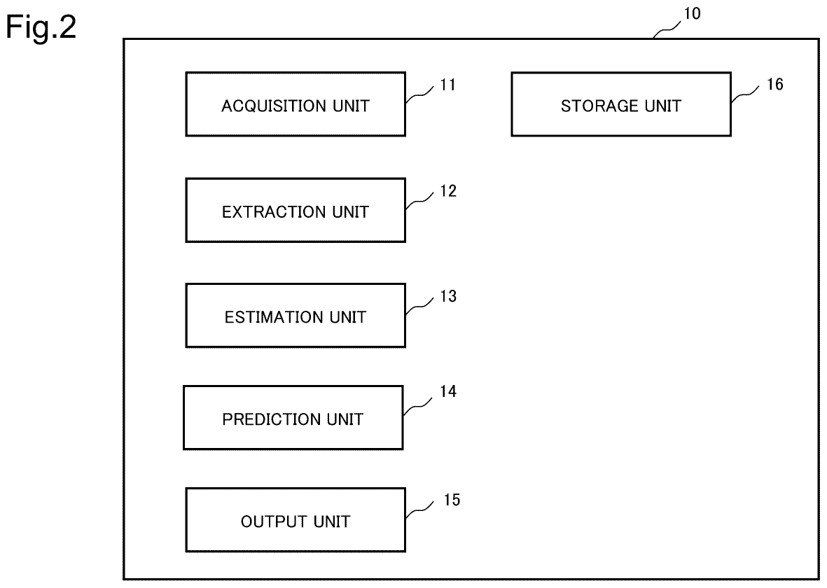

Here, a specific example of the configuration of the clinical trial support device 10 will be described. FIG. 2 is an example of a configuration of the clinical trial support device 10. The clinical trial support device 10 includes an acquisition unit 11, an extraction unit 12, an estimation unit 13, a prediction unit 14, and an output unit 15 as a basic configuration. The clinical trial support device 10 further includes, for example, a storage unit 16.

The acquisition unit 11 acquires data regarding treatment of a patient. The acquisition unit 11 acquires, for example, data regarding treatment of a patient suffering from a disease to be studied. The disease to be studied is, for example, a disease to be administered with a medicine to be verified by a clinical trial. In a case where a plurality of medicines are administered in order, the acquisition unit 11 may acquire data regarding treatment for a patient administered with a medicine before the medicine to be studied. For example, in a case where a clinical trial of an anticancer agent is conducted, the acquisition unit 11 may acquire data regarding treatment of a patient undergoing the previous-line treatment. The line is, for example, a stage of treatment. In the case of treatment of cancer, the line indicates, for example, the order of treatment by administration of an anticancer agent. A previous-line treatment refers, for example, to treatment with a medicine administered one stage before the medicine to be studied.

The data regarding treatment is, for example, a record of one or more items of diagnosis, examination, medication, surgery, follow-up, and patient state. The data regarding treatment may also include attributes of the patient. The attributes of the patient are, for example, patient states that do not change with treatment. The attributes of the patient are, for example, information of one or more items of age, sex, weight, height, occupation, previous disease, previous disease of the family, race, and place of residence. The attributes of the patient are not limited to the above.

The patient state includes, for example, information about one or more items of disease information, complication information, biomarkers, disease status, guideline scores, therapeutic effects, and test results. The therapeutic effect is, for example, an effect by treatment, administration of a drug, and surgery. The effect is not limited to the above. The test result is, for example, a result of a biological test, a diagnostic imaging test, and a genome test. The test result is not limited to the above. The data regarding the treatment may include information about a person in charge who has performed the medical practice. The information about the person in charge who has performed the medical practice is, for example, information indicating a medical worker who has performed the medical practice on the patient. The information indicating the medical worker who has performed the medical practice on the patient is, for example, a name or an identifier of a doctor, a nurse, a pharmacist, and a physical therapist.

The acquisition unit 11 acquires, for example, data of the electronic medical record as data regarding treatment of a patient. The data regarding the treatment of the patient may be test data. In a case where the data regarding treatment of the patient is test data, the data regarding treatment of the patient may be image data for image diagnosis. The data regarding treatment of the patient may be the data described in the receipt. The data regarding the treatment is not limited to the above.

The acquisition unit 11 may acquire extraction conditions of the clinical trial candidate. The acquisition unit 11 acquires, for example, information of one or more of items of a clinical trial period, a clinical department, a disease name, a target region, a criterion used for extraction of eligibility criteria, and a reference value of suitability as an extraction condition. The clinical trial period is, for example, information designating a period during which the clinical trial is carried out. The clinical department is, for example, information for designating a clinical department that treats a disease to be a target of a clinical trial. The target disease name is, for example, information designating a disease to be treated by administering a medicine to be studied. The clinical trial implementation region is, for example, information designating a region where a hospital that implements the clinical trial is located. The criterion used for extraction of the eligibility criteria is, for example, information designating a criterion used for extraction of a clinical trial candidate among the criteria including the eligibility criteria. The reference value of the suitability is, for example, information indicating a reference value in a case where the candidate is extracted as a clinical trial candidate in a case where the suitability is equal to or more than the reference value. The suitability is, for example, an index indicating a degree of matching of the data regarding the treatment to the eligibility criterion. The extraction conditions are not limited to the above.

The extraction unit 12 extracts a clinical trial candidate who is a candidate of a patient to be studied based on the data regarding the treatment. The extraction unit 12 extracts, for example, a patient that matches the eligibility criteria at the first time point as a clinical trial candidate based on the data regarding the treatment. The first time point is, for example, a clinical trial implementation protocol creation time point. For example, the extraction unit 12 extracts a patient whose data regarding the treatment matching the eligibility criteria as a clinical trial candidate from among patients for which the data regarding the treatment has been acquired. Matching to the eligibility criteria means, for example, that the data regarding the treatment satisfies each condition included in the eligibility criteria.

For example, the extraction unit 12 may extract a patient that matches the eligibility criteria from the first time point to the second time point as a clinical trial candidate based on data regarding treatment. For example, the extraction unit 12 extracts, as a clinical trial candidate, a patient that matches the eligibility criteria between the first time point and the second time point based on data regarding the treatment of the patient from among patients who are performing the previous-line treatment. The extraction unit 12 may extract a patient that matches the eligibility criteria from the second time point to the end of the clinical trial period as a clinical trial candidate. The extraction unit 12 may extract the clinical trial candidate based on the extraction condition acquired by the acquisition unit 11. For example, by extracting a clinical trial candidate based on an extraction condition set by a person in charge of selecting a clinical trial patient, a clinical trial candidate can be extracted under an optimized condition.

Eligibility criteria are, for example, inclusion criteria for patients into a clinical trial. The eligibility criteria may be patient exclusion criteria from the clinical trial. The eligibility criteria may be both inclusion criteria and exclusion criteria. The inclusion criteria are, for example, information indicating the conditions of the patient to be the subject of the clinical trial patient. The inclusion criteria are also referred to as eligibility criteria. Conditions of the patient to be the subject of the clinical trial patient are indicated using data regarding the treatment appropriate for the clinical trial patient. The exclusion criterion is information indicating a condition for excluding a patient from the target of the clinical trial patient. That is, the exclusion criterion is, for example, information indicating a condition of a patient not selected as a clinical trial patient. Exclusion criteria are indicated using data regarding the treatment of patients who are not suitable as clinical trial patients.

For example, the extraction unit 12 extracts, as a patient that matches the eligibility criteria, a patient whose data regarding treatment satisfies each criterion included in the eligibility criteria in the items designated by the eligibility criteria. For example, the extraction unit 12 extracts a patient whose data regarding the treatment is included within the range of the index designated in each of the items designated by the eligibility criteria. In a case where a criterion to be used for extracting a clinical trial candidate is selected among the eligibility criteria, the extraction unit 12 may extract a clinical trial candidate using, for example, a criterion selected among the eligibility criteria.

The extraction unit 12 may extract the patient based on the suitability of the data regarding the treatment with respect to the eligibility criteria. For example, the extraction unit 12 outputs a patient having a suitability with respect to the eligibility criteria of a reference value or more as a patient that matches the eligibility criteria. For example, the extraction unit 12 calculates a ratio of the number of criteria satisfied by the data regarding the treatment to the total number of criteria included in the eligibility criteria as the suitability.

The extraction unit 12 may calculate the suitability of each patient based on the weight of each item of the eligibility criteria. The extraction unit 12 may calculate the suitability of each patient based on the difference between the criteria included in the eligibility criteria and the data regarding the treatment of each patient and the weight of each item of the eligibility criteria. The weight of each item of the eligibility criteria is set, for example, based on the magnitude of the influence each of the items of the eligibility criteria can have on the result of the clinical trial. For example, in a case where the effect of the medicine to be tested is greatly affected by the administration history of other medicines, the weight of each item of the eligibility criteria is set such that the weight of the administration history of other medicines is larger than that of the other items.

The extraction unit 12 may extract a patient that matches the eligibility criteria using an extraction model. The extraction unit 12 extracts a patient that matches the eligibility criteria using the extraction model based on, for example, data regarding treatment of the patient and the eligibility criteria. The extraction model is, for example, a machine learning model that determines the presence or absence of matching the eligibility criteria using the eligibility criteria and data regarding the treatment as inputs. The extraction unit 12 extracts, for example, as clinical trial candidates, patients whom an extraction model has determined to be suitable for eligibility criteria. For example, the extraction model determines a patient having data regarding treatment similar to the eligibility criteria as a patient that matches the eligibility criteria. The extraction model is generated by deep learning using a neural network, for example. The extraction model is generated, for example, in an information processing device outside the clinical trial support device 10. The extraction model may be generated, for example, in a learning means (not illustrated) in the clinical trial support device 10.

The extraction unit 12 may extract data related to the eligibility criteria from the data regarding treatment using the language model. For example, the extraction unit 12 extracts a patient that matches the eligibility criteria based on the data extracted from the data regarding the treatment using the language model. As the language model, for example, a large language model is used. For example, Generative Pre-trained Transformer-2 (GPT-2), GPT-3, GPT-3.5, or GPT-4 can be used as the language model. Claude3, Claude3.5, text-to-text transfer transformer (T5), bidirectional encoder representations from transformers (BERT), robustly optimized BERT approach (RoBERTa), or efficiently learning an encoder that classifies token replacements accurately (ELECTRA) may be used as the language model. The language model used for the processing of extracting the data related to the eligibility criteria from the data regarding treatment is not limited to the above.

The extraction unit 12 may extract a patient that matches the eligibility criteria in a plurality of stages. For example, the extraction unit 12 extracts patients that match the eligibility criteria in two stages. For example, the extraction unit 12 extracts a patient based on a first criterion that is a criterion having a high priority among the eligibility criteria. The criterion having a high priority is, for example, a criterion that needs to be always satisfied by the patient for the purpose of the clinical trial. Then, the extraction unit 12 extracts a patient that matches the eligibility criteria based on a second criterion other than the first criterion among the eligibility criteria from among the patients extracted based on the first criterion. The second criterion is, for example, a criterion by which a patient who satisfies relaxed conditions can be suitable for the clinical trial. The second criterion may be a criterion by which the patient can be suitable for the clinical trial without determining whether the patient matches the criterion.

The extraction unit 12 may extract, as a clinical trial candidate, a patient that matches a criterion in which conditions included in the eligibility criterion for selecting a clinical trial candidate have been changed. Changing a part of the eligibility criteria means, for example, relaxing a condition in a part of a plurality of criteria included in the eligibility criteria. Relaxing the condition means, for example, widening the range of patients indicated by the criteria. For example, in a case where the changed criterion is age, relaxing the criterion means making the condition of 45 to 55 years old to 45 to 60 years old. Changing a part of the eligibility criteria may mean making the criteria stricter for some of the plurality of items included in the eligibility criteria. Changing a part of the eligibility criteria may include changing one criterion in multiple stages. Making the criteria stricter means, for example, narrowing the range of patients indicated by the criteria. Changing a part of the eligibility criteria may mean deleting a part of the plurality of criteria included in the eligibility criteria.

The data regarding treatment may be data subjected to pseudonymization processing. For example, the extraction unit 12 may extract a patient that matches the eligibility criteria from the data regarding the treatment subjected to pseudonymization processing. The data regarding the treatment subjected to pseudonymization processing is, for example, data in which information for specifying each patient is deleted, but each patient can be specified by collating other information. The data regarding the treatment may be subjected to anonymization processing. The data regarding the treatment subjected to anonymization processing is, for example, data in which information for specifying each patient is deleted and each patient cannot be specified even if the data is collated with other information.

The estimation unit 13 estimates a matching probability that is a probability that the clinical trial candidate is suitable for the clinical trial at a second time point after the first time point by using a state curve that is a curve indicating a time-series state change of a patient suffering from the disease to be studied. The state curve indicating the state change of the patient is time-series data on the state change of the patient who has suffered from a disease in the past and has been treated for the disease. The state curve is, for example, a Kaplan-Meier curve in the disease to be studied. The estimation unit 13 selects a state curve based on, for example, a disease and a stage of treatment. The estimation unit 13 selects a state curve based on the disease, the degree of progression of the disease, and the stage of treatment. The applicable state curve may be designated by the person in charge performing the extraction of the clinical trial candidate.

The estimation unit 13 estimates the probability of being suitable as the clinical trial patient at the second time point starting from the time point at which each patient satisfies the eligibility criteria for the clinical trial. The probability of being suitable as the clinical trial patient decreases with the lapse of time from the time point at which the eligibility criteria are satisfied. Therefore, for example, in a case where estimation is performed using the same state curve, the probability of being suitable as the clinical trial patient at the second time point is different between patients having different time points at which the eligibility criteria are satisfied. For example, for each patient, the estimation unit 13 estimates the probability of being suitable as the clinical trial patient at the second time point using the same state curve starting from the time point at which each patient satisfies the eligibility criteria of the clinical trial.

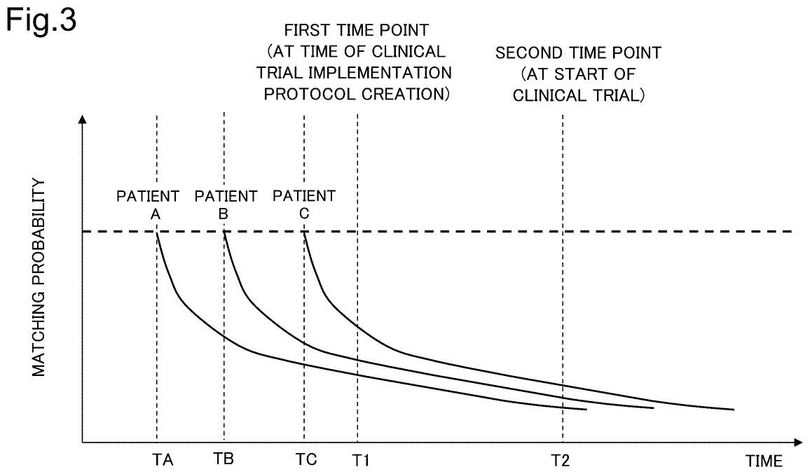

FIG. 3 is a graph schematically illustrating an example of a change in the state of each patient estimated using the state curve. In the example of the graph of FIG. 3, the vertical axis represents the matching probability, and the horizontal axis represents time. The example of the graph of FIG. 3 estimates changes in the state of three patients, Patient A, Patient B, and Patient C, using the state curves. The patient A, the patient B, and the patient C may refer to, for example, an individual patient or one or more patients having a specific attribute.

For example, it is assumed that patient A, patient B, and patient C are extracted as clinical trial candidates at the time point T₁ which is the first time point. The first time point is, for example, at the time of creation of the clinical trial protocol. In this case, for example, it is assumed that the patient A satisfies the eligibility criteria at the time point TA before the time point T₁. At this time, for example, in a case where the patient A refers to a patient with a specific attribute, the patient A is a group of patients satisfying the eligibility criteria at the time point TA. For example, it is assumed that the patient B satisfies the eligibility criteria at a time point T_{B} before the time point T₁. For example, it is assumed that the patient C satisfies the eligibility criteria at a time point Tc before the time point T₁. At this time, for example, the estimation unit 13 applies the state curve starting from the time point TA for the patient A to estimate the probability of being suitable as the clinical trial patient at the time point T₂ that is the second time point. The second time point is, for example, the start of the clinical trial. For example, the estimation unit 13 estimates the probability of being suitable as the clinical trial patient at the time point T₂ by applying the state curve starting from the time point T_{B} for the patient B. For example, the estimation unit 13 estimates the probability of being suitable as the clinical trial patient at the time point T₂ by applying the state curve starting from the time point T_{B} for the patient C. In this way, for example, the estimation unit 13 applies the same state curve starting from the time point at which each patient satisfies the eligibility criteria of the clinical trial, and estimates the probability of being suitable as the clinical trial patient at the start of the clinical trial. In a case where the patients A, B, and C are one or more patients with a specific attribute, the number of clinical trial candidates at the start of the clinical trial can be predicted by calculating the number of people in the group x the matching probability. For example, the number of clinical trial candidates at the start of the clinical trial in the group of patients A can be predicted, for example, by calculating (the number of people belonging to patient A)× matching probability.

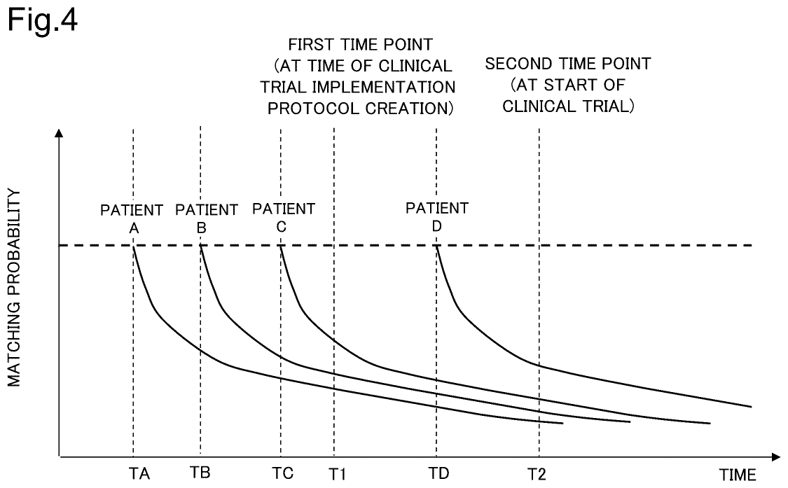

FIG. 4 is a graph schematically illustrating an example of a change in the state of the patient satisfying the eligibility criteria between the first time point and the second time point in the example of FIG. 3. In the example of the graph of FIG. 4, the patient D satisfies the eligibility criteria at the time point T_{D} between the time point T₁ and the time point T₂. For example, also for the patient D, the estimation unit 13 applies the state curve starting from the time point T_{D} to estimate the probability of being suitable as the clinical trial patient at the time point T₂.

The prediction unit 14 predicts the number of clinical trial candidates at the second time point based on the matching probability. For example, the prediction unit 14 predicts the number of patients having a matching probability equal to or more than the reference value at a predetermined time point as the number of clinical trial candidates at the predetermined time point. The reference value of the matching probability is set, for example, so that in a case where the matching probability is equal to or more than the reference value, the matching probability becomes a value that can be expected as a clinical trial candidate at a predetermined point in time.

The prediction unit 14 may calculate the sum of the matching probabilities of the patients at the second time point and predict the calculated sum as the number of clinical trial candidates at the second time point. The prediction unit 14 may weight the matching probability of the patient at the predetermined time point to calculate a sum, and predict the calculated sum as the number of clinical trial candidates at the second time point.

The prediction unit 14 may predict the number of clinical trial candidates at the second time point for each hospital to be subjected to the clinical trial. The prediction unit 14 predicts, for example, the number of clinical trial candidates at the second time point in each hospital, thereby predicting the number of clinical trial candidates at the second time point in each hospital to be subjected to the clinical trial.

The prediction unit 14 predicts, for example, a combination of hospitals in which the total number of clinical trial candidates satisfies the number necessary for implementation of the clinical trial. For example, the prediction unit 14 predicts a combination of hospitals capable of securing the number of clinical trial candidates necessary for implementation of the clinical trial by performing weighting based on predetermined criteria for each hospital.

The predetermined criteria are set based on, for example, one or more items of the presence or absence of a principal investigator of a clinical trial, the number of doctors, the scale of the hospital, the clinical trial performance, and the establishment form of the hospital. For example, in a case where the predetermined criterion is a criterion based on the presence or absence of a principal investigator of a clinical trial, the predetermined criterion is set such that the weighting becomes larger in a hospital in which the principal investigator of the clinical trial is present. For example, in a case where the predetermined criterion is a criterion based on the scale of the hospital, the predetermined criterion is set such that the weighting becomes larger as the scale of the hospital is larger. The scale of the hospital is, for example, at least one of the number of doctors and the number of beds for hospitalization in the clinical department that has jurisdiction over the disease to be studied. The scale of the hospital is not limited to the above. For example, in a case where the predetermined criterion is a criterion based on clinical trial performance, the predetermined criterion is set such that the weighting becomes larger in a hospital having a larger clinical trial performance. For example, in a case where the predetermined criterion is a criterion based on the establishment form, the predetermined criterion is set such that the weighting becomes larger in a hospital having higher expertise in advanced medical care such as a university hospital. How to set the predetermined criteria is not limited to the above.

The prediction unit 14 may predict the number of patients who will satisfy the eligibility criteria after the second time point as the number of additional patients. For example, the prediction unit 14 predicts the number of patients who will satisfy the eligibility criteria after the start of the clinical trial as the number of additional patients. In a case where the number of additional patients is predicted, the prediction unit 14 predicts the number of clinical trial candidates after the second time point based on, for example, the number of patients who are clinical trial candidates at the second time point and the number of additional patients.

The prediction unit 14 may predict the number of clinical trial candidates introduced from a hospital other than the target for predicting the number of clinical trial candidates as the number of introduced patients. In a case of predicting the number of introduced patients, the prediction unit 14 predicts the number of clinical trial candidates at the second time point based on, for example, the number of patients who are clinical trial candidates at the second time point among the clinical trial candidates at the first time point, the number of additional patients, and the number of introduced patients.

The prediction unit 14 may predict the number of clinical trial candidates at the second time point based on information regarding the clinical trial implementation competitor. For example, in a case where a competitor schedules a clinical trial for the same type of medicine, the prediction unit 14 multiplies the number of clinical trial candidates predicted based on the matching probability by a predetermined coefficient to predict the number of clinical trial candidates at the second time point. The predetermined coefficient is set based on, for example, a prediction result in a case where there is a competitor in a past clinical trial and the number of actually acquired clinical trial patients. The prediction unit 14 may predict the number of clinical trial candidates at the second time point by subtracting a predetermined number of people from the number of clinical trial candidates predicted based on the matching probability. The predetermined number of people is set based on, for example, a difference between a prediction result in a case where there is a competitor in a past clinical trial and the number of actually acquired clinical trial patients. The predetermined coefficient and the predetermined number of people may be set based on at least one of a scale of the competitor, a clinical trial performance at a hospital where the competitor's clinical trial is conducted, and a nationality of the competitor. The predetermined coefficient and the predetermined number of people can be appropriately set.

The output unit 15 outputs information regarding the number of clinical trial candidates at the second time point. For example, the output unit 15 outputs information regarding the number of clinical trial candidates at the start of the clinical trial. The output unit 15 may output the number of clinical trial candidates for each hospital. The output unit 15 may output a combination of hospitals in which the number of clinical trial candidates satisfies the number of patients required for the clinical trial.

The output unit 15 may output the expectation of the increased number of clinical trial candidates at the second time point. For example, the output unit 15 outputs, for example, the expectation of the increased number of clinical trial candidates after the start of the clinical trial for each hospital. The output unit 15 outputs, for example, the expectation of the increased number of clinical trial candidates from the start to the end of the clinical trial for each hospital. For example, the output unit 15 outputs the number of patients to be studied after the previous-line treatment ends after the start of the clinical trial as the expectation of the increased number of clinical trial candidates. The output unit 15 may output information indicating when a clinical trial candidate satisfying the eligibility criteria satisfies the eligibility criteria after the second time point. The output unit 15 may output the number of clinical trial candidates expected to increase due to introduction from hospitals.

The output unit 15 may output the amount of change in the number of clinical trial candidates for each changed condition in a case where the condition included in the eligibility criteria are changed. For example, the output unit 15 outputs an increase amount in the number of clinical trial candidates for each relaxed condition in a case where the condition included in the eligibility criteria is relaxed.

The output unit 15 may output a display screen for inputting a setting value related to the prediction processing of the number of clinical trial candidates. For example, the output unit 15 outputs, for example, a display screen for inputting extraction conditions of clinical trial candidates as a display screen for inputting a setting value regarding prediction processing of the number of clinical trial candidates. The output unit 15 outputs, for example, a display screen including an input for inputting information of one or more of items among a clinical trial period, a clinical department, a disease name, a target region, a criterion used for extraction of eligibility criteria, and a reference value of suitability. The output unit 15 outputs, for example, a display screen for inputting a setting value related to prediction processing of the number of clinical trial candidates to the terminal device 20.

The output unit 15 may output a display screen that displays the number of clinical trial candidates superimposed on a map based on the prediction result of the number of clinical trial candidates. The output unit 15 may display a screen for making an inquiry about the clinical trial candidate to the hospital where the clinical trial candidate is undergoing treatment. The output unit 15 may output a display screen displaying a list of clinical trial candidates who are undergoing treatment in each hospital. In a case where a hospital is selected on the display screen of the number of clinical trial candidates for each hospital, the output unit 15 may output a display screen displaying a list of clinical trial candidates undergoing treatment in the selected hospital. The output unit 15 may output a display screen displaying at least one of the state of the clinical trial candidate and the data regarding treatment in a case where the clinical trial candidate is selected from the list of the clinical trial candidates. The output unit 15 may output a display screen displaying a history of treatment of a clinical trial candidate in a case where the clinical trial candidate is selected from the list of the clinical trial candidates. The output unit 15 may output a display screen displaying extraction conditions used for extracting the selected clinical trial candidate. The output unit 15 may output a display screen displaying information indicating the criteria used for extracting the selected clinical trial candidate among the eligibility criteria.

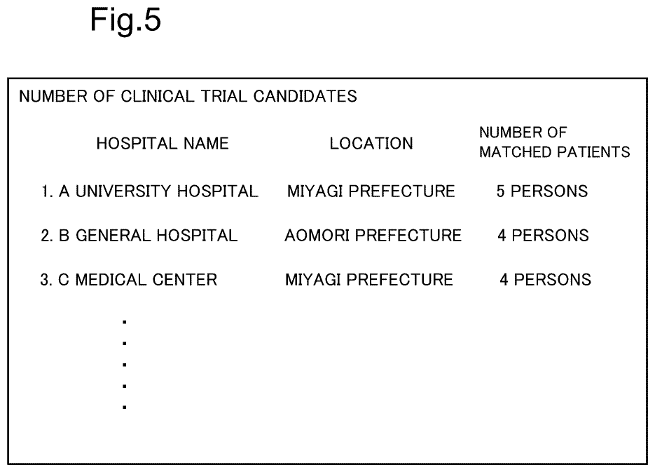

FIG. 5 is an example of a display screen displaying a prediction result of the number of clinical trial candidates. In the example of the display screen of FIG. 5, the prediction result of the number of clinical trial candidates is displayed in the form of a list of the number of people for each hospital. In the example of the display screen of FIG. 5, hospitals are displayed in order of a larger number of clinical trial candidates. For example, if it is possible to grasp hospitals that can secure as many candidate patients as possible in a case of conducting a clinical trial, the number of hospitals to be contacted can be reduced. Therefore, by displaying the hospitals in descending order of the number of clinical trial candidates, for example, it is possible to improve the efficiency of the operation related to the implementation of the clinical trial. The criteria regarding the order of displaying the number of clinical trial candidates for each hospital can be appropriately set.

In the example of the display screen of FIG. 5, “hospital name”, “location”, and “number of matched patients” are displayed in association with each other. The “hospital name” is, for example, a name of a hospital. The “location” is, for example, a place where the hospital is located. In the example of the display screen of FIG. 5, the “location” is displayed in units of prefectures. The display of “location” is not limited to the units of prefecture. The “number of matched patients” is, for example, the number of clinical trial candidates. The “location” may be displayed in the order of the hospitals where the installation place is closer to the position information of the terminal device 20. The “location” may group and display hospitals close to each other in installation location. For example, in a case where the hospital A, the hospital C, and the hospital D are located in places close to each other, and the hospital B, the hospital E, and the hospital F are located in places close to each other, the list may be displayed as a group of the hospital A, the hospital C, and the hospital D, and the hospital B, the hospital E, and the hospital F from the top of the list. For example, a company that conducts a clinical trial may dispatch a person in charge to a hospital to perform operations related to implementation of the clinical trial. Therefore, by grasping the hospitals located at positions close to each other, it is possible to improve the efficiency of the operations related to the implementation of the clinical trial, for example.

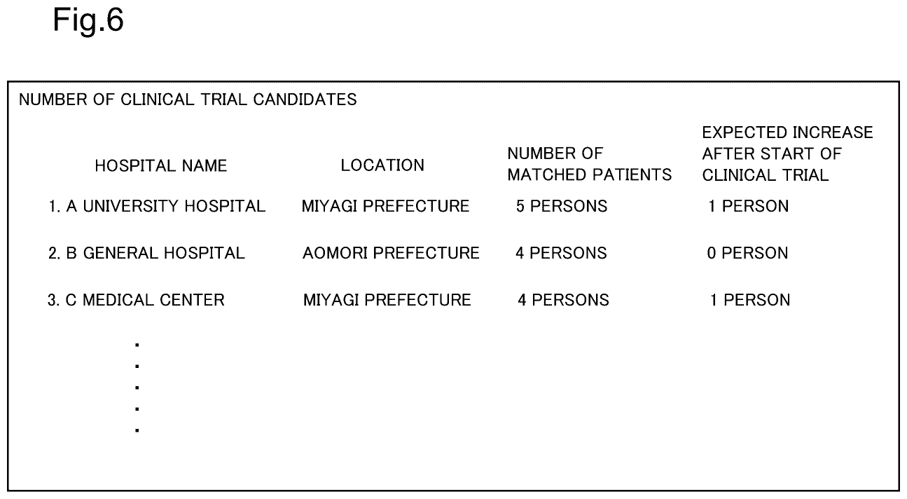

FIG. 6 is an example of a display screen displaying the prediction result of the number of clinical trial candidates including the clinical trial candidates that increase after the start of the clinical trial. In the example of the display screen of FIG. 6, the prediction result of the number of clinical trial candidates at the start of the clinical trial and the prediction result of the number of clinical trial candidates after the start of the clinical trial are displayed in the form of a list of the number of people for each hospital. In the example of the display screen of FIG. 6, hospitals are displayed in order of a larger number of clinical trial candidates. In the example of the display screen of FIG. 6, “hospital name”, “location”, “number of matched patients”, and “expected increase after start of clinical trial” are associated with each hospital. The “expected increase after start of clinical trial” is, for example, the number of patients who will satisfy the eligibility criteria after the start of the clinical trial.

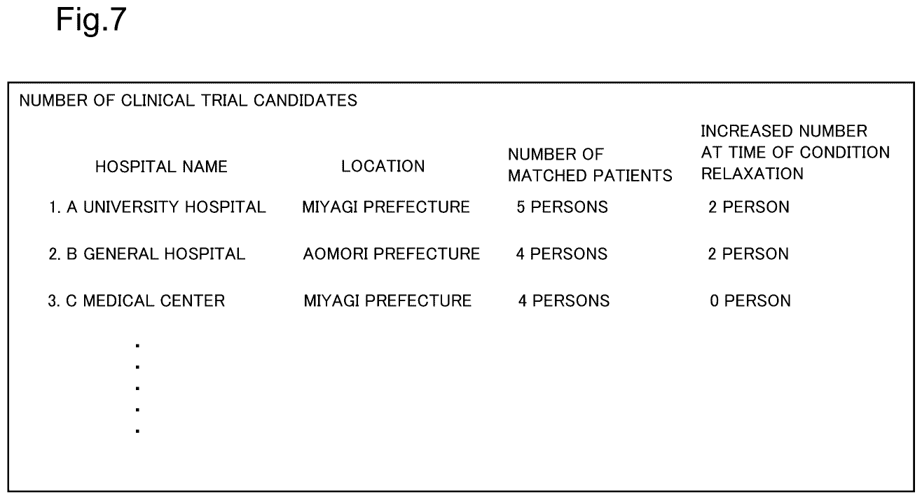

FIG. 7 is an example of a display screen displaying the prediction result of the number of clinical trial candidates in a case where the eligibility criteria are relaxed. In the example of the display screen of FIG. 7, the prediction result of the number of clinical trial candidates satisfying the eligibility criteria and the prediction result of the number of clinical trial candidates in a case where the eligibility criteria are relaxed are displayed in the form of a list of the number of people for each hospital. In the example of the display screen of FIG. 7, hospitals are displayed in order of a larger number of clinical trial candidates. In the example of the display screen of FIG. 7, “hospital name”, “location”, “number of matched patients”, and “increased number at the time of condition relaxation” are associated with each hospital. The “increased number at the time of condition relaxation” is, for example, the number of patients who newly satisfy the eligibility criteria in a case where part of the criteria included in the eligibility criteria is relaxed.

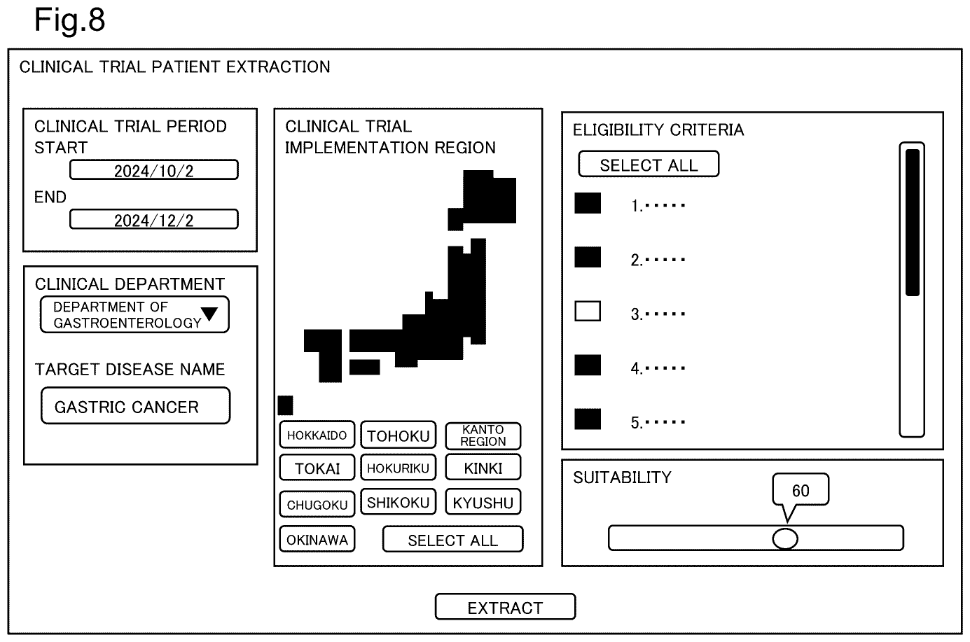

FIG. 8 is an example of a display screen for setting an extraction condition for extracting a clinical trial candidate. In the example of the display screen of FIG. 8, “clinical trial period”, “clinical department”, “target disease name”, “clinical trial implementation region”, “eligibility criteria”, and “suitability” are displayed as setting items. In the example of the display screen of FIG. 8, the “clinical trial period” is, for example, a setting field of a period for performing a clinical trial. In the example of the display screen of FIG. 8, in the setting field of the “clinical trial period”, “start” which is a field for inputting the first day of the period and “end” which is a field for inputting the last day of the period are displayed. In the example of the display screen of FIG. 8, “clinical department” is, for example, a field for selecting a clinical department in charge of a disease to which a medicine to be studied is administered. A plurality of “clinical departments” may be selected. In the example of the display screen of FIG. 8, the “target disease name” is, for example, a field for inputting a disease to which a medicine to be studied is administered.

In the example of the display screen of FIG. 8, “clinical trial implementation region” is, for example, a field for selecting a region to be a clinical trial target. In the example of the display screen of FIG. 8, a button for selecting a region in which a clinical trial is conducted is displayed in the “clinical trial implementation region”. In the example of the display screen of FIG. 8, the “clinical trial implementation region” is selected in local units, for example. The “clinical trial implementation region” may be selected, for example, in units of prefectures.

In the example of the display screen of FIG. 8, the “eligibility criteria” is, for example, a field for selecting a criterion to be used for extraction of a clinical trial candidate among the eligibility criteria. In the example of the display screen of FIG. 8, a black square indicates a criterion selected. In the example of the display screen of FIG. 8, a white square indicates a criterion not selected. In the example of the display screen of FIG. 8, “suitability” is used as a criterion for extraction as a clinical trial candidate, for example, in a case where suitability for treatment with respect to the eligibility criterion exceeds a set value. In the example of the display screen of FIG. 8, the “suitability” is set by moving a slider. The “suitability” may be set by inputting a numerical value. In the example of the display screen of FIG. 8, an “extract” button is displayed at the bottom. The “extract” button is, for example, a button for instructing execution of extraction processing of a clinical trial candidate using an extraction condition.

The output unit 15 outputs a display screen of the extraction condition as illustrated in FIG. 8 to the terminal device 20, for example. Then, for example, the acquisition unit 11 acquires, from the terminal device 20, the extraction condition input by the operation of the person in charge on the display screen displayed by the terminal device 20. The person in charge is, for example, a person who creates a clinical trial implementation protocol using the prediction result of the number of clinical trial candidates. The person in charge is not limited to the above. When the “extract” button is pressed by the operation of the person in charge on the display screen, the acquisition unit 11 acquires, for example, information indicating that the “extract” button has been pressed. When the information indicating that the “extract” button has been pressed is acquired, the extraction unit 12 extracts a clinical trial candidate using, for example, an extraction condition set in a case where the button has been pressed.

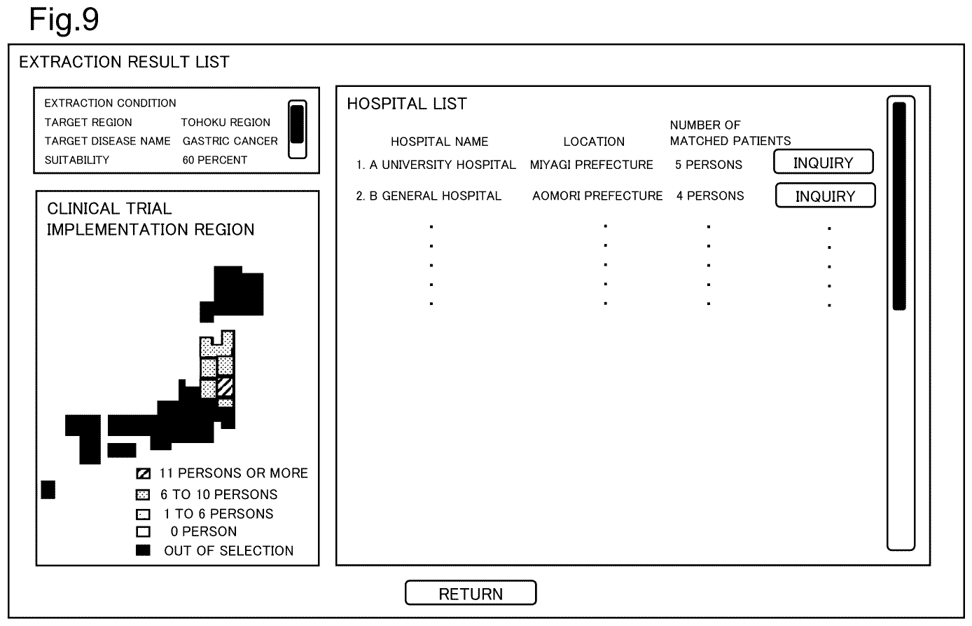

FIG. 9 is an example of a display screen displaying a prediction result of the number of clinical trial candidates. In the example of the display screen of FIG. 9, “extraction condition”, “clinical trial implementation region”, and “hospital list” are displayed. In the example of the display screen of FIG. 9, the “extraction condition” is a field for displaying a condition for extracting a clinical trial candidate. In the example of the display screen of FIG. 9, the “extraction condition” is, for example, a condition input on the display screen as illustrated in FIG. 8. In the example of the display screen of FIG. 9, the “clinical trial implementation region” is, for example, a region where a target hospital from which the clinical trial candidate is extracted is located. In the “clinical trial implementation region” in the example of the display screen of FIG. 9, the number of clinical trial candidates is superimposed and displayed on the map. In the example of the display screen of FIG. 9, the “hospital list” indicates a prediction result of the number of clinical trial candidates for each hospital. In the “hospital list” in the example of the display screen of FIG. 9, “hospital name”, “location”, and “number of matched patients” are displayed in association with each other. The “hospital name” is, for example, a name of a hospital. The “location” is, for example, a place where the hospital is located. In the example of the display screen of FIG. 9, the “location” is displayed in units of prefectures. The display of “location” is not limited to the units of prefecture. The “number of matched patients” is, for example, the number of clinical trial candidates. In the example of the display screen of FIG. 9, an “inquiry” button is displayed for each hospital. When the button of “inquiry” is pressed in the example of the display screen of FIG. 9, the output unit 15 outputs a screen for contacting the selected hospital, for example.

In the example of the display screen of FIG. 9, a “secure patient” button may be further displayed. For example, the output unit 15 outputs, to the terminal device 20, a display screen further displayed by a “secure patient” button on a display screen as illustrated in FIG. 9. Then, for example, the acquisition unit 11 acquires, from the terminal device 20, information indicating that the “secure patient” button has been pressed by the operation of the person in charge on the display screen displayed by the terminal device 20. When the information indicating that the “secure patient” button has been pressed is acquired, the output unit 15 collectively outputs a message requesting securing clinical trial candidates to the hospital listed, for example. The message requesting the securing of the clinical trial candidate may be selectable for each hospital.

In the example of the display screen of FIG. 9, the terminal device 20 may automatically execute the processing of requesting the hospitals listed to secure the clinical trial candidates without the user's operation in accordance with the display of the display screen. Specifically, the terminal device 20 acquires a hospital list based on the prediction result of the number of clinical trial candidates, automatically selects a transmission target hospital according to a predetermined policy (for example, the necessary secured number, the priority region, the hospital attribute, and the weighting), and collectively transmits a message for requesting each selected hospital to secure clinical trial candidates. The selection of the transmission target may be automatically performed by the terminal device 20 for each hospital, and the transmission result may be recorded by the terminal device 20 and displayed as necessary.

The terminal device 20 automatically starts the securing request transmission processing with the display of the display screen as a trigger, and eliminates the waiting time caused by the user operation waiting. As a result, the end-to-end delay until the request message reaches each hospital can be shortened, and variations in the delay can also be suppressed. The terminal device 20 may automatically record an audit log including a transmission target, a transmission time, an identifier of a transmission message, a delivery confirmation result, the presence or absence of retry, and the like for each transmission processing. This ensures traceability of the transmission history, and can facilitate consistency verification and failure analysis at a later date.

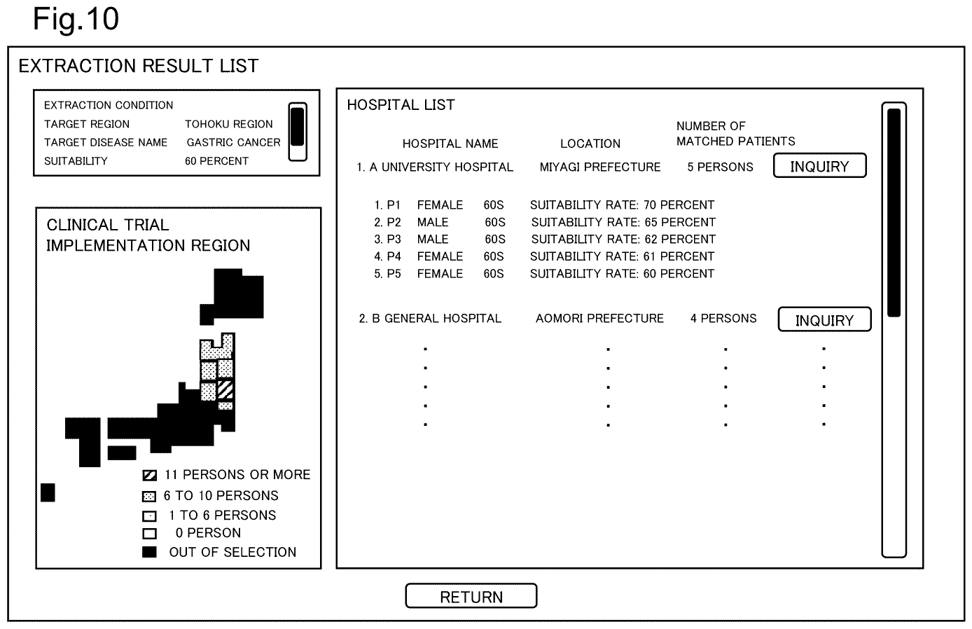

FIG. 10 is an example of a display screen for displaying a list of clinical trial candidates in a selected hospital in a case where the hospital is selected in the example of the display screen of FIG. 9. In the example of the display screen of FIG. 10, “extraction condition”, “clinical trial implementation region”, and “hospital list” are displayed. In the “hospital list” in the example of the display screen of FIG. 10, a list of clinical trial candidates of “A university hospital” is displayed. For example, in a case where any hospital is selected in the “hospital list” in the example of the display screen of FIG. 9 displayed on the display device of the terminal device 20, the acquisition unit 11 acquires the hospital selection result from the terminal device 20. Then, the output unit 15 outputs, for example, a list of clinical trial candidates in the selected hospital based on the selection result.

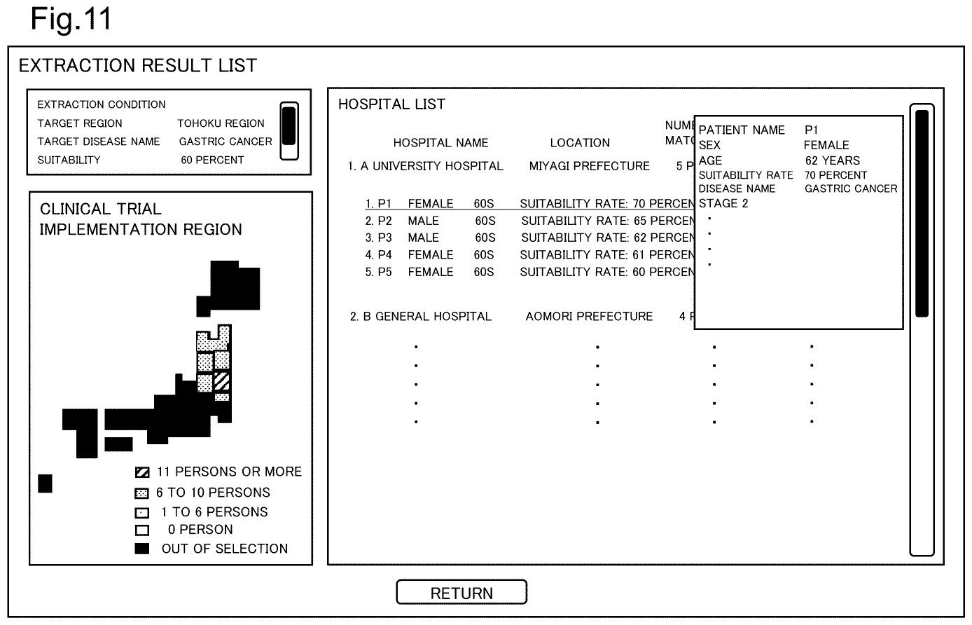

FIG. 11 is an example of a display screen that displays at least part of data regarding treatment of a selected clinical trial candidate in a case where the clinical trial candidate is selected from the list of the clinical trial candidates in the example of the display screen of FIG. 10. For example, in a case where any one of the clinical trial candidates is selected in the list of the clinical trial candidates in the example of the display screen of FIG. 10, the output unit 15 outputs at least part of the data regarding the treatment of the selected clinical trial candidate. In the example of the display screen of FIG. 11, data regarding the treatment of the patient P1 selected in the example of the display screen of FIG. 10 is displayed in a pop-up format. For example, in a case where the cursor is moved to the position of any hospital in the list of the clinical trial candidates in the example of the display screen of FIG. 10 displayed on the display device of the terminal device 20 and the clinical trial candidate is selected, the acquisition unit 11 acquires the selection result of the clinical trial candidate from the terminal device 20. Then, for example, the output unit 15 outputs, to the terminal device 20, a display screen displaying data regarding the treatment of the selected clinical trial candidate in a pop-up format based on the selection result, for example.

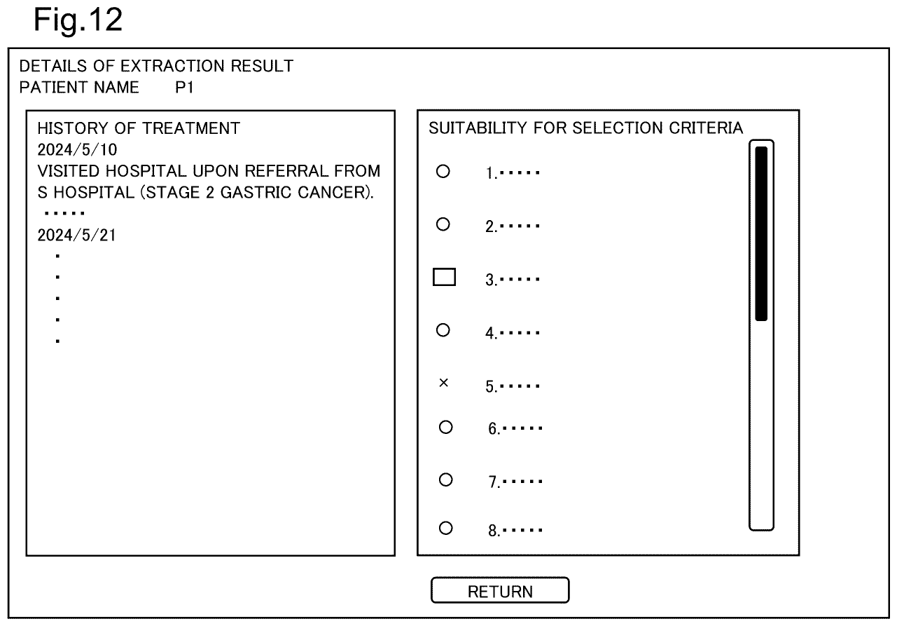

FIG. 12 is an example of a display screen for displaying detailed information of a selected clinical trial candidate in a case where the clinical trial candidate is selected from the list of the clinical trial candidates in the example of the display screen of FIG. 10. In the example of the display screen of FIG. 12, “patient name”, “history of treatment”, and “suitability for eligibility criteria” of the selected clinical trial candidate are displayed. In the example of the display screen of FIG. 10, the “patient name” is a name or identifier of a patient. In the example of the display screen of FIG. 10, the “history of treatment” is information indicating the progress of treatment performed on the patient. In the example of the display screen of FIG. 10, the “suitability for eligibility criteria” is information indicating the presence or absence of the suitability of the data regarding the treatment for each criterion included in the eligibility criteria. In the example of the display screen of FIG. 10, “o” indicates that the data regarding the treatment satisfies the criterion. In the example of the display screen of FIG. 10, “x” indicates that the data regarding the treatment does not satisfy the criteria. In the example of the display screen of FIG. 10, a white square indicates a criterion that is not selected as a criterion to be used for extraction of a clinical trial candidate. For example, in a case where any clinical trial candidate is selected in the patient list of the example of the display screen of FIG. 10 displayed on the display device of the terminal device 20, the acquisition unit 11 acquires the selection result of the clinical trial candidate from the terminal device 20. Then, the output unit 15 outputs, for example, a display screen displaying detailed information of the selected clinical trial candidate on the terminal device 20 based on the selection result.

The storage unit 16 stores, for example, data related to prediction of the number of clinical trial candidates. The storage unit 16 stores, for example, data regarding treatment of a patient. The storage unit 16 stores, for example, an eligibility criterion. The storage unit 16 stores, for example, extraction conditions of the clinical trial candidate. The storage unit 16 stores, for example, the extracted identification information of the clinical trial candidate and data regarding the treatment of the clinical trial candidate in association with each other. The storage unit 16 stores, for example, the estimation result of the matching probability at the second time point of the clinical trial candidate. The storage unit 16 stores the prediction result of the number of clinical trial candidates at the second time point. The storage unit 16 stores, for example, the extraction model. The extraction model may be stored in a storage means other than the storage unit 16.

The terminal device 20 may be, for example, an information processing device used for processing of accessing the clinical trial support device 10 and extracting a clinical trial candidate. The terminal device 20 is, for example, a terminal device used by a person in charge of a medical institution or a person in charge of an institution commissioned by the medical institution to conduct the operation related to clinical trials. The institution to which the hospital entrusts handling of the clinical trial is, for example, a site management organization (SMO). The person in charge of an institution commissioned by a medical institution to conduct the operation related to clinical trials is, for example, a clinical research coordinator (CRC). The terminal device 20 may be a terminal device used by a person in charge of a clinical trial at a pharmaceutical company or a person in charge at an institution commissioned by the pharmaceutical company to carry out a clinical trial of a medicine. The institution commissioned with the clinical trial by the pharmaceutical company is, for example, a contract research organization (CRO). The person in charge of the institution commissioned with the clinical trial by the pharmaceutical company is, for example, a clinical research associate (CRA).

The terminal device 20 may be an information processing device used by a medical worker to grasp the number of clinical trial candidates. The medical worker is, for example, a doctor, a nurse, a pharmacist, a clinical laboratory technician, a physical therapist, a counselor, a hospital clerical staff member, or a hospital consultant. The medical worker is not limited to the above.

The terminal device 20 acquires, for example, information regarding the number of clinical trial candidates from the output unit 15 of the clinical trial support device 10. Then, the terminal device 20 outputs information regarding the number of clinical trial candidates to a display device (not illustrated), for example. For example, the terminal device 20 acquires a display screen displaying the number of clinical trial candidates for each hospital from the output unit 15 of the clinical trial support device 10 as information regarding the number of clinical trial candidates. Then, the terminal device 20 outputs a display screen displaying the number of clinical trial candidates for each hospital to a display device (not illustrated), for example.

In a case where a hospital is selected on the display screen displaying the number of clinical trial candidates for each hospital, the terminal device 20 outputs information indicating the selected hospital to the acquisition unit 11 of the clinical trial support device 10, for example. The terminal device 20 acquires a display screen displaying a list of clinical trial candidates in the hospital relevant to the selection result from the output unit 15 of the clinical trial support device 10, for example. Then, the terminal device 20 outputs a display screen displaying a list of clinical trial candidates to a display device (not illustrated), for example.

In a case where a clinical trial candidate is selected on the display screen displaying the list of the clinical trial candidates, the terminal device 20 outputs information indicating the clinical trial candidate to the acquisition unit 11 of the clinical trial support device 10, for example. For example, the terminal device 20 acquires a display screen displaying detailed information about the clinical trial candidate relevant to the selection result from the output unit 15 of the clinical trial support device 10. Then, the terminal device 20 outputs a display screen displaying detailed information of the clinical trial candidate to a display device (not illustrated), for example.

The terminal device 20 acquires, for example, a display screen for setting extraction conditions of the clinical trial candidate from the output unit 15 of the clinical trial support device 10. Then, the terminal device 20 outputs a display screen for setting extraction conditions of the clinical trial candidate to a display device (not illustrated), for example. The terminal device 20 outputs, to the acquisition unit 11 of the clinical trial support device 10, the extraction conditions of the clinical trial candidate input by the operation of the person in charge on the display screen for setting the extraction conditions of the clinical trial candidate.

As the terminal device 20, for example, a personal computer, a tablet computer, a smartphone, or a smartwatch can be used. The information processing device used for the terminal device 20 is not limited to the above.

The data management device 30 stores, for example, data regarding treatment of a patient. The data management device 30 stores, for example, data of the electronic medical record as data regarding treatment of a patient. The data regarding the treatment of the patient may be test data. In a case where the data regarding treatment of the patient is test data, the data regarding treatment of the patient may be image data for image diagnosis. The data regarding treatment of the patient may be the data described in the receipt. The data regarding treatment may be, for example, recognition of a disease state by a patient. The data management device 30 outputs data regarding treatment to the acquisition unit 11 of the clinical trial support device 10, for example.

The data management device 30 stores, for example, data regarding treatment of a patient in each hospital that accepts implementation of a clinical trial. The data management device 30 may be located for each hospital and store data regarding treatment of a patient in the located hospital. The data management device 30 may be located for each group of hospitals, and may store data regarding treatment of patients in a hospital belonging to the group. How the data management device 30 stores the data regarding the treatment can be appropriately set.

For example, the data management device 30 may store data regarding treatment as anonymization processing information or pseudonymization processing information. The anonymization processing information is, for example, information processed so that an individual cannot be specified even if the anonymization processing information is collated with other information. The pseudonymization processing information is, for example, information processed so that an individual cannot be specified by itself but can be specified by collating with other information. The data management device 30 may perform anonymization processing or pseudonymization processing in a case where outputting the data regarding the treatment to the acquisition unit 11 of the clinical trial support device 10 and output the data regarding the treatment.

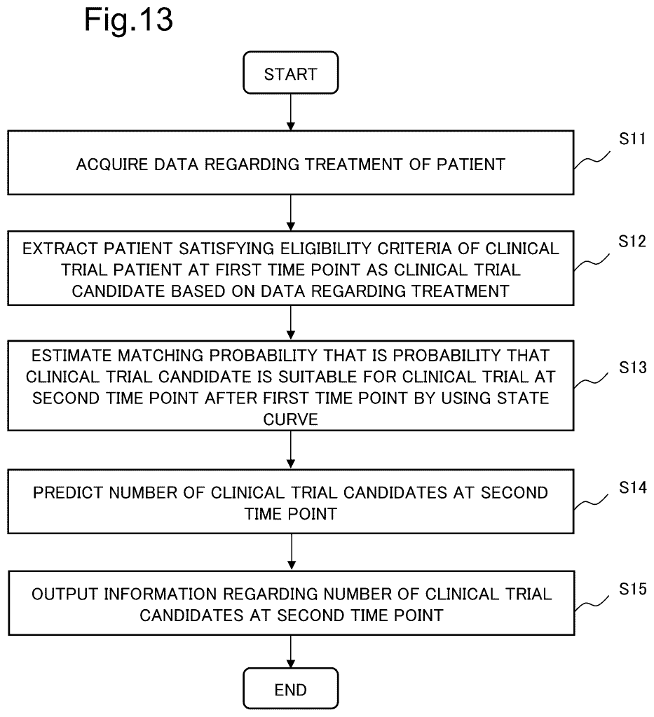

Processing in which the clinical trial support device 10 predicts the number of clinical trial candidates at the second time point will be described. FIG. 13 is an example of an operation flow in the processing in which the clinical trial support device 10 predicts the number of clinical trial candidates at the second time point.

The acquisition unit 11 acquires data regarding treatment of a patient (step S11). The acquisition unit 11 acquires, for example, data regarding treatment of a patient from the data management device 30.

When the data regarding the treatment of the patient is acquired, the extraction unit 12 extracts a patient satisfying the eligibility criteria of the clinical trial patient at the first time point as a clinical trial candidate based on the data regarding the treatment (step S12).

Once the clinical trial candidate is extracted, the estimation unit 13 estimates a matching probability, which is the probability that the clinical trial candidate is suitable for the clinical trial at a second time point after the first time point, using the state curve (step S13). The state curve is a curve illustrating a time-series state change of a patient suffering from a disease to be studied.

After estimating the matching probability, the prediction unit 14 predicts the number of clinical trial candidates at the second time point based on the matching probability (step S14).

When the number of clinical trial candidates at the second time point is predicted, the output unit 15 outputs information regarding the number of clinical trial candidates at the second time point (step S15). The output unit 15 outputs, for example, information regarding the number of clinical trial candidates at the second time point to the terminal device 20.

Each processing in the clinical trial support device 10 may be executed in a distributed manner in a plurality of information processing devices connected via a network. For example, the processing in the extraction unit 12 and the processing in the estimation unit 13 and the prediction unit 14 may be performed in different information processing devices. Which information processing device performs each processing in the clinical trial support device 10 can be appropriately set.

The clinical trial support device 10 extracts a patient who satisfies the eligibility criteria of the clinical trial patient at the first time point as the clinical trial candidate based on the data regarding the treatment. The clinical trial support device 10 estimates a matching probability, which is a probability that the clinical trial candidate is suitable for the clinical trial at a second time point after the first time point, using a state curve that is a curve indicating a time-series state change of a patient suffering from the disease to be studied. Then, the clinical trial support device 10 outputs information regarding the number of clinical trial candidates at the second time point. By predicting the number of clinical trial candidates in this manner, the clinical trial support device 10 can improve the accuracy of prediction of the number of clinical trial candidates.

For example, assuming that the first time point is the time of creating the clinical trial implementation protocol and the second time point is the time of starting the clinical trial, the clinical trial support device 10 can accurately predict the number of clinical trial candidates at the time of starting the clinical trial based on the data regarding the treatment at the time of creating the clinical trial implementation protocol. By predicting the number of clinical trial candidates for each hospital, for example, a person in charge of selecting a clinical trial patient can accurately determine a hospital to negotiate for securing a clinical trial patient. Therefore, the clinical trial support device 10 can support, for example, decision-making in selection of a clinical trial patient.

By predicting the number of clinical trial candidates at the second time point including patients satisfying the condition as a clinical trial candidate between the first time point and the second time point, the clinical trial support device 10 can facilitate, for example, securing of the clinical trial patient. By further predicting the patient satisfying the condition as the clinical trial candidate after the second time point, the clinical trial support device 10 can more easily secure the clinical trial patient, for example. By predicting an increase or decrease in the number of clinical trial candidates in a case where the eligibility criteria are changed, the clinical trial support device 10 can increase the possibility of being able to secure the clinical trial patient even in a case where there is not a sufficient number of clinical trial candidates satisfying the eligibility criteria, for example.

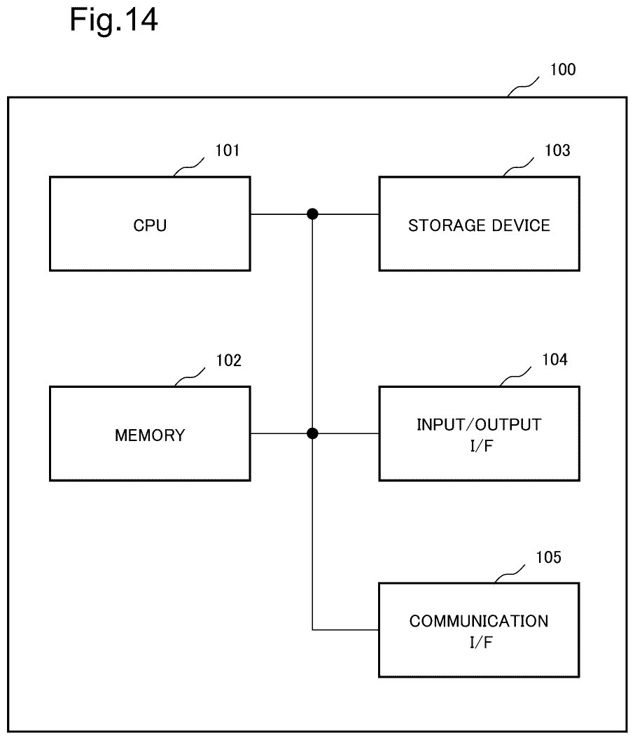

Each processing in the clinical trial support device 10 can be enabled by executing a computer program on a computer. FIG. 14 illustrates an example of a configuration of a computer 100 that executes a computer program for performing each processing in the clinical trial support device 10. The computer 100 includes a central processing unit (CPU) 101, a memory 102, a storage device 103, an input/output interface (I/F) 104, and a communication I/F 105.

The CPU 101 reads and executes a computer program for performing each processing from the storage device 103. The CPU 101 may be configured by a combination of a plurality of CPUs. The CPU 101 may be configured by a combination of a CPU and another type of processor. For example, the CPU 101 may be configured by a combination of a CPU and a graphics processing unit (GPU). The memory 102 includes a dynamic random access memory (DRAM) or the like, and temporarily stores a computer program executed by the CPU 101 and data being processed. The storage device 103 stores a computer program executed by the CPU 101. The storage device 103 includes, for example, a nonvolatile semiconductor storage device. As the storage device 103, another storage device such as a hard disk drive may be used. The input/output I/F 104 is an interface that receives an input from an operator and outputs a display screen and the like. The communication I/F 105 is an interface that transmits and receives data to and from the terminal device 20, the data management device 30, and other information processing devices. The terminal device 20 and the data management device 30 can also be configured similarly to the computer 100.

The computer program used for executing each processing can also be distributed by being stored in a computer-readable recording medium that non-transitory records data. As the recording medium, for example, a magnetic tape for data recording or a magnetic disk such as a hard disk can be used. As the recording medium, an optical disk such as a compact disc read only memory (CD-ROM) can also be used. A nonvolatile semiconductor storage device may be used as a recording medium.

In a pharmaceutical trial, a person in charge of selecting patients to be studied extracts patients that match the eligibility criteria of clinical trial patients, for example, by checking the description of medical records. For example, the person in charge determines whether the eligibility criteria match the contents described in the medical records. Then, the person in charge extracts patients whose eligibility criteria and the contents described in the medical record match as candidates for the patients to be studied. In order to secure the number of clinical trial patients required for the clinical trial, the person in charge needs to check the medical records of many patients and search for patients that match the eligibility criteria. For this reason, a system that supports selection of patients to be studied may be used to select patients to be studied.

The clinical study support system of WO 2023/248978 A1 screens a database storing clinical data based on eligibility criteria and exclusion criteria of a clinical study. Then, the clinical study support system of WO 2023/248978 A1 narrows down candidates for a clinical study based on a screening result.

In the technique described in WO 2023/248978 A1, it may be difficult to accurately predict the number of clinical trial candidates.

In order to solve the above problems, an object of the present disclosure is to provide a clinical trial support device and the like capable of improving the accuracy of prediction of the number of clinical trial candidates.

According to the present disclosure, the accuracy of prediction of the number of clinical trial candidates can be improved.

Some or all of the above example embodiments may be described as the following supplementary notes, but are not limited to the following.

### Supplementary Note 1

A clinical trial support device including:
an acquisition unit that acquires data regarding treatment of a patient;
an extraction unit that extracts, as a clinical trial candidate, a patient who satisfies eligibility criteria of a clinical trial patient at a first time point based on the data regarding the treatment;
an estimation unit that estimates a matching probability that is a probability that the clinical trial candidate is suitable for the clinical trial at a second time point after the first time point by using a state curve that is a curve indicating a time-series state change of a patient suffering from a disease to be studied;
a prediction unit that predicts the number of clinical trial candidates at the second time point based on the matching probability; and
an output unit that outputs information regarding the number of clinical trial candidates at the second time point.

### Supplementary Note 2

The clinical trial support device according to Supplementary Note 1, wherein
the prediction unit predicts the number of clinical trial candidates at the second time point for each hospital to be subjected to a clinical trial.

### Supplementary Note 3

The clinical trial support device according to Supplementary Note 2, wherein
the prediction unit predicts a combination of hospitals in which the total number of clinical trial candidates satisfies the number required to perform the clinical trial.

### Supplementary Note 4

The clinical trial support device according to Supplementary Note 3, wherein
the prediction unit predicts a combination of hospitals capable of securing the number of the clinical trial candidates necessary for implementation of the clinical trial by performing weighting based on predetermined criteria for each of the hospitals.

### Supplementary Note 5

The clinical trial support device according to any one of Supplementary Notes 1 to 4, wherein
the prediction unit predicts the number of patients who will satisfy eligibility criteria after the second time point as the number of additional patient, and predicts the number of clinical trial candidates after the second time point based on the number of patients who are clinical trial candidates at the second time point and the number of the additional patients.

### Supplementary Note 6

The clinical trial support device according to any one of Supplementary Notes 1 to 5, wherein
the extraction unit extracts, as the clinical trial candidate, a patient that matches a criterion in which a condition included in the eligibility criteria is relaxed.

### Supplementary Note 7

The clinical trial support device according to Supplementary Note 5, wherein
the prediction unit predicts the number of clinical trial candidates introduced from a hospital other than a target for predicting the number of clinical trial candidates as the number of introduced patients, and predicts the number of clinical trial candidates after the second time point based on the number of patients who are clinical trial candidates at the second time point among the clinical trial candidates at the first time point, the number of the additional patients, and the number of the introduced patients.

### Supplementary Note 8

The clinical trial support device according to any one of Supplementary Notes 1 to 7, wherein
the prediction unit predicts the number of the clinical trial candidates at the second time point based on information regarding a clinical trial implementation competitor.

### Supplementary Note 9

The clinical trial support device according to Supplementary Note 5, wherein
the output unit outputs information indicating when a clinical trial candidate who will satisfy the eligibility criteria after the second time point satisfies the eligibility criteria.

### Supplementary Note 10

The clinical trial support device according to Supplementary Note 6, wherein
the output unit outputs an increase amount of the number of clinical trial candidates for each relaxed condition in a case where the condition included in the eligibility criteria is relaxed.

### Supplementary Note 11

The clinical trial support device according to any one of Supplementary Notes 1 to 10, wherein
the state curve is a Kaplan-Meier curve in the disease to be studied.

### Supplementary Note 12

The clinical trial support device according to any one of Supplementary Notes 1 to 11, wherein
the eligibility criteria include inclusion criteria to the clinical trial and exclusion criteria from the clinical trial.

### Supplementary Note 13

The clinical trial support device according to any one of Supplementary Notes 1 to 12, wherein
the extraction unit extracts a subject as the clinical trial candidate using a machine learning model that determines whether the subject meets the eligibility criteria using the eligibility criteria and the data regarding the treatment as inputs.

### Supplementary Note 14

The clinical trial support device according to any one of Supplementary Notes 1 to 13, wherein
the output unit outputs, to a terminal device, a display screen for selecting a criterion to be used for extraction of the clinical trial candidate among the eligibility criteria, the acquisition unit acquires, from the terminal device, a criterion selected on the display screen displayed by the terminal device, and the extraction unit extracts a patient satisfying the criterion selected as the clinical trial candidate.

### Supplementary Note 15

The clinical trial support device according to any one of Supplementary Notes 1 to 14, wherein
the output unit outputs, to a terminal device, a display screen for setting a reference value of suitability indicating a degree to which the data regarding the treatment satisfies the eligibility criteria,
the acquisition unit acquires, from the terminal device, a reference value set on the display screen displayed on the terminal device, and
the extraction unit extracts a patient whose suitability is equal to or more than the reference value set as the clinical trial candidate.

### Supplementary Note 16

The clinical trial support device according to any one of Supplementary Notes 1 to 15, wherein
the output unit outputs a display screen displaying the number of clinical trial candidates of each hospital on a terminal device,
the acquisition unit acquires a hospital selected on the display screen displayed on the terminal device from the terminal device, and
the output unit outputs a list of the clinical trial candidates in the hospital selected to the terminal device.

### Supplementary Note 17

The clinical trial support device according to Supplementary Note 16, wherein
the acquisition unit acquires, from the terminal device, a clinical trial candidate selected on the display screen of the list of the clinical trial candidates displayed on the terminal device, and
the output unit outputs at least a part of the data regarding the treatment of the clinical trial candidate selected to the terminal device.

### Supplementary Note 18

The clinical trial support device according to Supplementary Note 17, wherein
the output unit outputs, to the terminal device, information indicating whether the data regarding the treatment of the clinical trial candidate selected satisfies each of the eligibility criteria.

### Supplementary Note 19

A clinical trial support method including:
acquiring data regarding treatment of a patient;
extracting, as a clinical trial candidate, a patient who satisfies eligibility criteria of a clinical trial patient at a first time point based on the data regarding the treatment;
estimating a matching probability that is a probability that the clinical trial candidate is suitable for the clinical trial at a second time point after the first time point by using a state curve that is a curve indicating a time-series state change of a patient suffering from a disease to be studied;
predicting the number of clinical trial candidates at the second time point based on the matching probability; and
outputting information regarding the number of clinical trial candidates at the second time point.

### Supplementary Note 20

A recording medium non-transitorily recording a program for causing a computer to execute:
a process of acquiring data regarding treatment of a patient;
a process of extracting, as a clinical trial candidate, a patient who satisfies eligibility criteria of a clinical trial patient at a first time point based on the data regarding the treatment;
a process of estimating a matching probability that is a probability that the clinical trial candidate is suitable for the clinical trial at a second time point after the first time point by using a state curve that is a curve indicating a time-series state change of a patient suffering from a disease to be studied;
a process of predicting the number of clinical trial candidates at the second time point based on the matching probability; and
a process of outputting information regarding the number of clinical trial candidates at the second time point.

Some or all of the configurations described in Supplementary Notes 2 to 18 dependent on the above-described Supplementary Note 1 can also depend on Supplementary Notes 19 and 20 by the same dependency relationship as Supplementary Notes 2 to 18. Furthermore, not only the Supplementary Notes 1, 19, and 20, but also various pieces of hardware, software, and various recording means or systems for recording software can be similarly dependent on some or all of the configurations described as the Supplementary Notes without departing from the above-described example embodiments.

The previous description of embodiments is provided to enable a person skilled in the art to make and use the present disclosure. Moreover, various modifications to these example embodiments will be readily apparent to those skilled in the art, and the generic principles and specific examples defined herein may be applied to other embodiments without the use of inventive faculty. Therefore, the present disclosure is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. A clinical trial support device comprising:
at least one memory storing instructions; and
at least one processor configured to access the at least one memory and execute the instructions to:
acquire data regarding treatment of a patient;
extract, as a clinical trial candidate, a patient who satisfies eligibility criteria of a clinical trial patient at a first time point based on the data regarding the treatment;
estimate a matching probability that is a probability that the clinical trial candidate is suitable for the clinical trial at a second time point after the first time point by using a state curve that is a curve indicating a time-series state change of a patient suffering from a disease to be studied;
predict the number of clinical trial candidates at the second time point based on the matching probability; and
output information regarding the number of clinical trial candidates at the second time point.

2. The clinical trial support device according to claim 1, wherein
the at least one processor is further configured to execute the instructions to: predict the number of clinical trial candidates at the second time point for each hospital to be subjected to a clinical trial.

3. The clinical trial support device according to claim 2, wherein
the at least one processor is further configured to execute the instructions to:
predict a combination of hospitals in which the total number of clinical trial candidates satisfies the number required to perform the clinical trial.

4. The clinical trial support device according to claim 3, wherein
the at least one processor is further configured to execute the instructions to:
predict a combination of hospitals capable of securing the number of the clinical trial candidates necessary for implementation of the clinical trial by performing weighting based on predetermined criteria for each of the hospitals.

5. The clinical trial support device according to claim 1, wherein
the at least one processor is further configured to execute the instructions to:
predict the number of patients who will satisfy eligibility criteria after the second time point as the number of additional patients, and predict the number of clinical trial candidates after the second time point based on the number of patients who are clinical trial candidates at the second time point and the number of the additional patients.

6. The clinical trial support device according to claim 1, wherein
the at least one processor is further configured to execute the instructions to:
extract, as the clinical trial candidate, a patient that matches a criterion in which a condition included in the eligibility criteria is relaxed.

7. The clinical trial support device according to claim 5, wherein
the at least one processor is further configured to execute the instructions to:
predict the number of clinical trial candidates introduced from a hospital other than a target for predicting the number of clinical trial candidates as the number of introduced patients, and predicts the number of clinical trial candidates after the second time point based on the number of patients who are clinical trial candidates at the second time point among the clinical trial candidates at the first time point, the number of the additional patients, and the number of the introduced patients.

8. The clinical trial support device according to claim 1, wherein
the at least one processor is further configured to execute the instructions to:
predict the number of the clinical trial candidates at the second time point based on information regarding a clinical trial implementation competitor.

9. The clinical trial support device according to claim 5, wherein
the at least one processor is further configured to execute the instructions to:
output information indicating when a clinical trial candidate who will satisfy the eligibility criteria after the second time point satisfies the eligibility criteria.

10. The clinical trial support device according to claim 6, wherein
the at least one processor is further configured to execute the instructions to:
output an increase amount of the number of clinical trial candidates for each relaxed condition in a case where the condition included in the eligibility criteria is relaxed.

11. The clinical trial support device according to claim 1, wherein
the state curve is a Kaplan-Meier curve in the disease to be studied.

12. The clinical trial support device according to claim 1, wherein
the eligibility criteria include inclusion criteria to the clinical trial and exclusion criteria from the clinical trial.

13. The clinical trial support device according to claim 1, wherein
the at least one processor is further configured to execute the instructions to:
extract a subject as the clinical trial candidate using a machine learning model that determines whether the subject meets the eligibility criteria using the eligibility criteria and the data regarding the treatment as inputs.

14. The clinical trial support device according to claim 1, wherein
the at least one processor is further configured to execute the instructions to:
output, to a terminal device, a display screen for selecting a criterion to be used for extraction of the clinical trial candidate among the eligibility criteria;
acquire, from the terminal device, a criterion selected on the display screen displayed by the terminal device; and
extract a patient satisfying the criterion selected as the clinical trial candidate.

15. The clinical trial support device according to claim 1, wherein
the at least one processor is further configured to execute the instructions to:
output, to a terminal device, a display screen for setting a reference value of suitability indicating a degree to which the data regarding the treatment satisfies the eligibility criteria;
acquire, from the terminal device, a reference value set on the display screen displayed on the terminal device; and
extract a patient whose suitability is equal to or more than the reference value set as the clinical trial candidate.

16. The clinical trial support device according to claim 1, wherein
the at least one processor is further configured to execute the instructions to:
output a display screen displaying the number of clinical trial candidates of each hospital on a terminal device;
acquire a hospital selected on the display screen displayed on the terminal device from the terminal device; and
output a list of the clinical trial candidates in the hospital selected to the terminal device.

17. The clinical trial support device according to claim 16, wherein
the at least one processor is further configured to execute the instructions to:
acquire, from the terminal device, a clinical trial candidate selected on the display screen of the list of the clinical trial candidates displayed on the terminal device; and
output at least a part of the data regarding the treatment of the clinical trial candidate selected to the terminal device.

18. The clinical trial support device according to claim 17, wherein
the at least one processor is further configured to execute the instructions to:
output, to the terminal device, information indicating whether the data regarding the treatment of the clinical trial candidate selected satisfies each of the eligibility criteria.

19. A clinical trial support method comprising:
acquiring data regarding treatment of a patient;
extracting, as a clinical trial candidate, a patient who satisfies eligibility criteria of a clinical trial patient at a first time point based on the data regarding the treatment;
estimating a matching probability that is a probability that the clinical trial candidate is suitable for the clinical trial at a second time point after the first time point by using a state curve that is a curve indicating a time-series state change of a patient suffering from a disease to be studied;
predicting the number of clinical trial candidates at the second time point based on the matching probability; and
outputting information regarding the number of clinical trial candidates at the second time point.

20. A non-transitory recording medium that records a program for causing a computer to execute:
a process of acquiring data regarding treatment of a patient;
a process of extracting, as a clinical trial candidate, a patient who satisfies eligibility criteria of a clinical trial patient at a first time point based on the data regarding the treatment;
a process of estimating a matching probability that is a probability that the clinical trial candidate is suitable for the clinical trial at a second time point after the first time point by using a state curve that is a curve indicating a time-series state change of a patient suffering from a disease to be studied;
a process of predicting the number of clinical trial candidates at the second time point based on the matching probability; and
a process of outputting information regarding the number of clinical trial candidates at the second time point.
